(19) United States
(12) Patent Application Publication
(10) Pub. No.: US 2006/0117399 A1
(43) Pub. Date: Jun. 1, 2006

(54) Animal model for human lymphomas

(75) Inventors: Han-Fei Ding, Perrysburg, OH (US); Baochun Zhang, Toledo, OH (US)

Correspondence Address: EMCH, SCHAFFER, SCHAUB & PORCELLO CO, P O BOX 916, ONE SEAGATE SUITE 1980, TOLEDO, OH, 43697, US

(73) Assignee: Medical University, Toledo, OH (US)

(21) Appl. No.: 11/290,192

(22) Filed: Nov. 29, 2005

Related U.S. Application Data
(60) Provisional application No. 60/631,779, filed on Nov. 30, 2004.

(51) Int. Cl. A01K 67/027

(52) U.S. Cl. 800/18

(57) ABSTRACT

This invention develops the first mouse model for human lymphomas with mutations in the NF-KB2 gene that can be used for the study of the pathogenesis of this subset of lymphomas and for the development and testing of therapeutic and prevention drugs. The mice develop B- and T-cell lymphomas with extensive metastases in the liver, lung and kidneys, and can be used as an animal model for the development and testing of therapeutic/prevention drugs that target this group of human lymphoma patients.

### CROSS REFERENCE

The present patent application is based upon and claims the benefit of provisional patent application No. 60/631,779 filed Nov. 30, 2004.

### TECHNICAL FIELD OF THE INVENTION

This invention relates to the field of cancer diagnosis and treatment. More particularly, this invention relates to using p80HT transgenic mice as an animal model for human lymphomas with NF-kB2 mutations. More specifically, the invention relates to perturbation of B cell homeostasis and lymphomagenesis in NF-_{K}B2 mutant transgenic mice.

### BACKGROUND OF THE INVENTION

The present invention is the building of large databases containing human genome sequences is the basis for studies of gene expressions in various tissues during normal physiological and pathologic conditions. Constantly (constitutively) expressed sequences as well as sequences whose expression is altered during disease processes are important for our understanding of cellular properties, and for the identification of candidate genes for future therapeutic intervention. As the number of known genes and ESTs build up in the databases, array-based simultaneous screening of thousands of genes is necessary to obtain a profile of transcriptional behavior, and to identify key genes that, either alone or in combination with other genes, control various aspects of cellular life. One cellular behavior that has been mystery for many years is the malignant behavior of cancer cells. We now know that, for example, defects in DNA repair can lead to cancer, but the cancer-creating mechanism in heterozygous individuals is still largely unknown, as is the malignant cell's ability to repeat cell cycles, to avoid apoptosis, to escape the immune system, to invade and metastasize, and to escape therapy. There are hints and indications in these areas and excellent progress has been made, but the myriad of genes interacting with each other in a highly complex multidimensional network is making the road to insight long and contorted.

Existing methods in the prior art regarding studies of NF-_{K}B2 mutants have been conducted in cells cultured in dishes (in vitro). Studies in cells cannot establish a casual relationship between NF-_{K}B2 mutations and lymphoma development. Also, cells cannot be used as a system to determine the therapeutic efficacy of anti-tumor drugs. Using P80HT transgenic mice provides the first animal model for studying human lymphomas with NF-_{K}B2 mutations.

### BRIEF SUMMARY OF THE INVENTION

This invention develops the first mouse model for human lymphomas with mutations in the NF-_{K}B2 gene that can be used for the study of the pathogenesis of this subset of lymphomas and for the development and testing of therapeutic and prevention drugs. We have developed the first mouse model for a subset of human lymphomas carrying specific genetic mutations. The mice develop B- and T-cell lymphomas with extensive metastases in the liver, lung and kidneys, and can be used as an animal model for the development and testing of therapeutic prevention drugs that target this group of human lymphoma patients. In this way, we can study the mice with the mutation rather than study human beings.

The NF-_{K}B2 gene is a member of the NF-_{K}B family that is recurrently mutated in human lymphoid malignancies. However, a casual relationship between the genetic alterations and lymphomagenesis remains to be established. Here we report the generation of transgenic mice with high0level constitutive _{K}B-binding activity in lymphocytes by targeted expression of p80HT, a lymphoma-associated NF-_{K}B2 mutant. The transgenic mice display a marked increase in the B cell population and develop predominantly B-cell lymphomas. p80HT expression has no apparent effect on the proliferation of B cells, but renders them specifically resistant to apoptosis induced by cytokine deprivation and mitogenic stimulation. Lymphocytes and lymphoma cells from p80HT transgenic mice express high levels of TRAF1, an anti-apoptotic protein also implicated in lymphoid malignancies. These findings demonstrate NF-_{K}B2 mutations as an oncogenic event in vivo and suggest distinct mechanisms for constitutive activation of NF-_{K}B in tumorigenesis.

Overwhelming evidence indicates a critical role of aberrant activation of NF-_{K}B in tumorigenesis initiated by oncogenes, inflammation and other defects in NF-_{K}B upstream signaling pathways. However, it is much less clear whether constitutive NF-_{K}B activation per se is sufficient to induce tumor development. Our study provides the first demonstration in vivo of a tumorigenic role of NF-_{K}B2 mutations, which occur recurrently in human lymphoid malignancies and product NF-_{K}B2 mutants that are constitutive transactivators. The finding also illustrates the concept that distinct mechanisms underlie the aberrant activation of NF-_{K}B1 and NF-_{K}B2 in tumorigenesis. The mouse model generated in this study should be useful in the development and testing of therapeutic and prevention drugs that target the subset of human lymphomas with NF-_{K}B2 mutations.

Other objects and advantages of the present invention will become apparent to those skilled in the art upon a review of the following detailed description of the preferred embodiments and the accompanying drawings.

### BRIEF DESCRIPTION OF THE DRAWINGS

FIG. 1 shows the characterization of p80HT transgenic mice.

FIG. 2 illustrates that p80HT expression leads to lymphoma development in transgenic mice.

FIG. 3 illustrates that p80HT transgenic mice that develop predominantly disseminated B lymphoma.

FIG. 4 provides data showing lymphocytes and lymphoma cells from p80HT transgenic mice are resistant to certain apoptotic stimuli.

FIG. 5 shows analyses of p80HT transgenic mice using Southern blot genotyping and Immunoblot analysis.

### DETAILED DESCRIPTION OF THE INVENTION

As the first animal model for the human lymphomas with NF-KB2 mutations, p80HT transgenic mice can be used for the study of the pathogenesis of this subject of lymphomas and for the development and testing of therapeutic and prevention drugs.

The mammalian NF-_{K}B family consists of five structurally related proteins including RelA, RelB, c-Rel, NF-_{K}B1 (p50 and its precursor p105), and NF-_{K}B2 (p52 and its precursor p100). The active forms of NF-_{K}B are dimeric complexes, composed of various combinations of family members, which bind a common DNA sequence motif known as the KB site and regulate the expression of genes crucial to the proper development and function of the immune system. NF-_{K}B activity is tightly controlled by I_{K}B (inhibitor _{K}B) proteins and the I_{K}B-like ankyrin-repeat domain in the C-terminal region of NF-_{K}B2 p100. I_{K}B proteins interact with NF-_{K}B dimmers composed of NF-_{K}B1 p50 and RelA or c-Rel, and NF-KB2 p100 is primarily associated with Relb. The interactions prevent NF-_{K}B dimmers from translocating to the nucleus, thereby maintaining them in an inactive state. Upon stimulation by certain cytokines, such as TNF-a and LT-β, IKBs and the C-terminal region of NF-_{K}B2 p100 are phosphorylated by the IKK (I_{K}B kinase) complex, which leads to proteasome-mediated degradation of I_{K}Bs or removal of the C-terminal ankyrin-repeat domain of NF-_{K}B2 p100. The freed NF-_{K}B1 p50-RelA/c-Rel or resulted NF-_{K}B2 p52-RelB dimmers then translocate to the nucleus and transactivate their target genes (Hayden and Ghosh, 2004; Li and Verma, 2002).

Constitutive NF_{-K}B activation is frequently observed in tumors and has been shown to play an important role in oncogenic development by promoting cell proliferation and by preventing apoptosis (Aggarwal, 2004; Karin et al., 2002). A number of mechanisms have been identified by which activation of NF-_{K}B is uncoupled from its normal modes of regulation in cancer cells. Most of these mechanisms target the IKK complex for activation of NF-_{K}B. For example, the Tax oncoprotein of human T-cell leukemia virus activates the IKK complex by direct interaction with the IKK_{Y} submit (Chu et al., 1999; Harhaj and Sun, 1999; Jin et al. 1999). Bcl10 and MALT1, which are implicated in the pathogenesis of mucosa-associated lymphoid tissue (MALT) lymphomas (Kkagi et al., 1999; Dierlamm et al., 1999; Morgan et al., 1999; Willis et al., 1999; Zhang et al., 1999), play an essential role in the activation of IKK in lymphocytes initiated by antigen-receptor stimulation (Lucas et al., 2001; Ruland et al., 2001; Uren et al. 2000). More recently, it has been shown that IKK-dependent activation of NF-_{K}B, triggered by proinflammatory cytokines, is essential for promoting inflammation-associated cancer in mouse model systems (Greten et al., 2004; Pikarski et al., 2004).

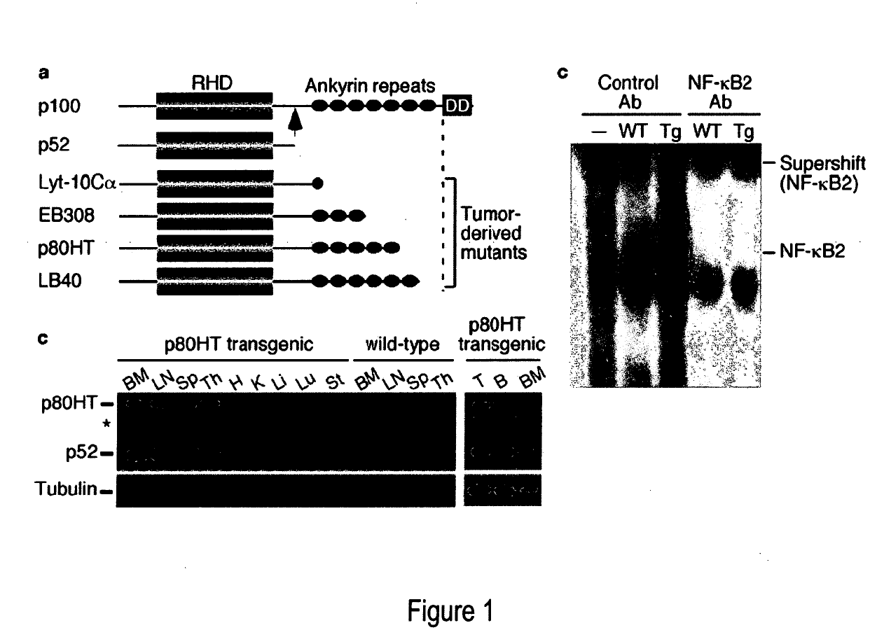

Constitutive NF-_{K}B activation can also be caused by genetic alterations that affect the activity and expression of the NF-_{K}B family of proteins (Rayet and Gelinas, 1999). The first gene of the family found to be mutated in human lymphoid malignancies is NF-_{K}B2, located at the chromosomal region 10q24 (Neri et al, 1991). Subsequent studies revealed that chromosomal rearrangements at the NF-_{K}B2 locus occur in a variety of B- and T-cell lymphoid malignancies (Fracchiolla et al., 1993; Migliazza et al., 1994; Thakur et al., 1994; Zhang et al., 1994). A cardinal feature of these genetic alterations is the generation of C-terminally truncated NF-_{K}B2 mutants that lack various portions of the ankyrin-repeat domain (FIG. 1A) and are constitutively active nuclear transactivators (Chang et al., 1995). Some of these mutants have been sown to be able to transform immortalized mouse fibroblasts (Balb/3T3) and the transformed cells gave rise to tumors in immunodeficient mice (Ciana et al., 1997), suggesting an oncogenic potential of NF-_{K}B2 mutants. However, the expression of these mutants had an apparent cytotoxic effect, which may explain their low transformation efficiency in mouse fibroblasts and failure to transform human lymphoblastoid cell lines immortalized by the Epstein-Barr virus (Ciana et al., 1997). This invention shows that NF-_{K}B2 mutations can directly initiate lymphomagenesis.

In this invention, we show that transgenic mice with targeted expression of a human lymphoma-associated NF-_{K}B2 mutant in lymphocytes develop lymphoma. This finding provides direct evidence that NF-_{K}B2 mutations play a causal role in the pathogenesis of lymphoid malignancies. This suggest that the NF-_{K}B2 mutant promotes lymphomagenesis by suppressing specific apoptotic responses critical for the maintenance of B cell homeostasis.

FIG. 1. Characterization of p80HT Transgenic Mice

A: Schematic diagram of NF-_{K}B2, p100, p52, and representative tumor-derived mutants. The arrow indicates the cleavage site on p100 that gives rise to p52. RHD, Relahomology domain; DD, death domain.

B: Immunoblot analysis of tissue and cell specific expression of p80HT, as well as of its processed product p52, using an antibody against the N-terminal region of human NF-_{K}B2. The star indicates a probably degraded p80HT product. Levels of α-tubulin are also shown as the loading control. BM, bone marrow; LN, lymph node; Sp, spleen; Th, thymus; H, heart; K, kidney; Li, liver; Lu, lung; St, stomach; T, splenic T cells; B, splenic B cells.

C: EMSA of nuclear KB-binding activity in splenic lymphocytes from p80HT transgenic (Tg) and wild-type (WT) mice. The position of the KB-binding complex containing NF-_{K}B2 (p80HT and P52) is indicated, which could be “supershifted” by an antibody against the N-terminal region of NF-_{K}B2. A non-specific antibody was used as the control.

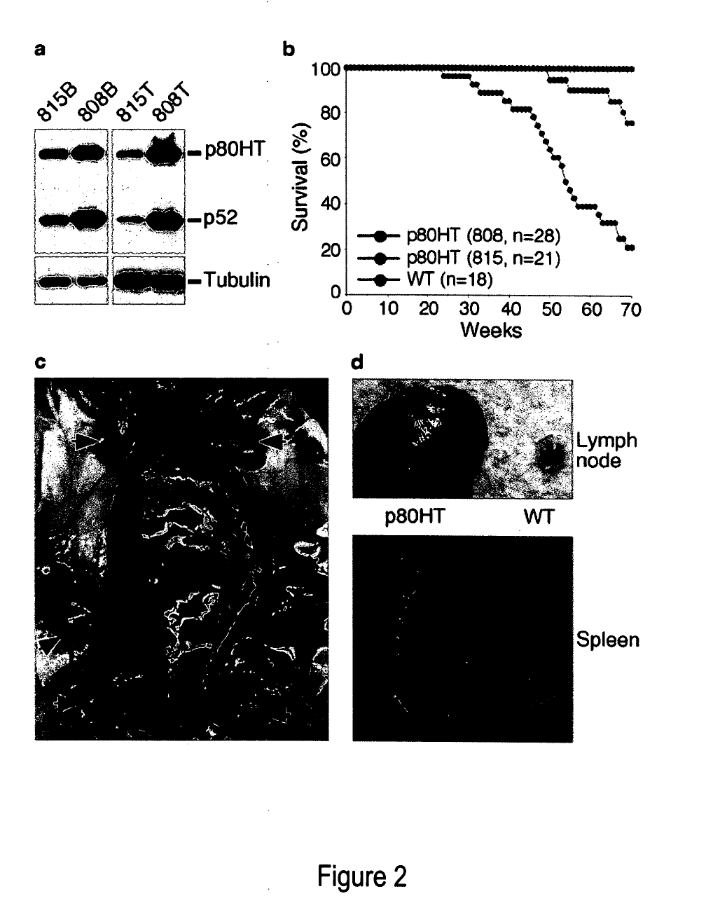

FIG. 2. p80HT Expression Leads to Lymphoma Development in Transgenic Mice.

A: Immunoblot analysis of p80HT (and p52) expression levels in splenic B cells (815B and 808B) and T cells (815T and 808T) from the 808 and 815 lines of transgenic mice. Levels of α-tubulin are also shown as the loading control.

B: Survival curve of p80HT transgenic mice (line 808 and 815) and their wild-type littermates. Numbers of mice for each group are indicated.

C: Autopsy examination of a deceased p80HT transgenic mouse showing markedly enlarged lymph nodes (arrows).

D: Representative examples of lymph nodes and spleens from a deceased p80HT transgenic mouse and an age-matched wild-type littermate.

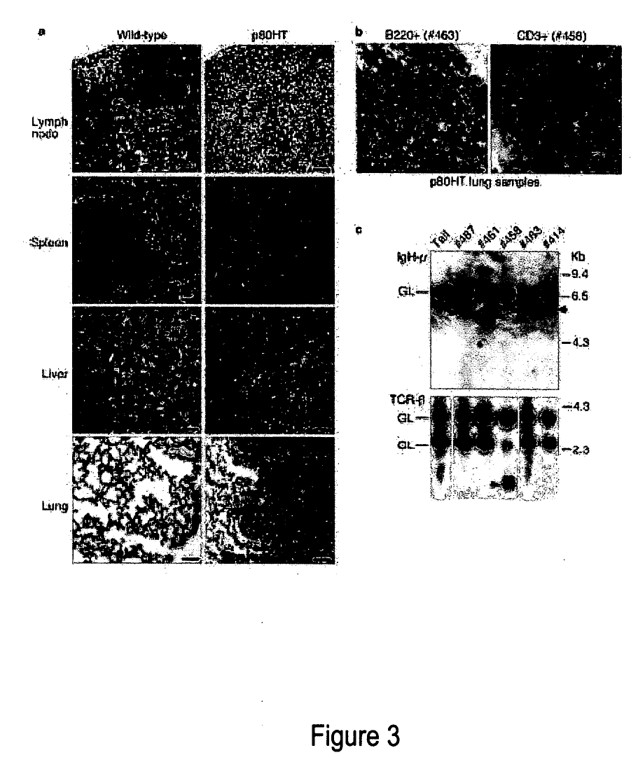

FIG. 3. p80HT Transgenic Mice Develop Predominantly Disseminated B Lymphoma.

A. Histopathological analysis of lymphomas in p80HT transgenic mice. Sections were stained with hematoxylin and eosin. The normal architecture shown in the wild-type organs are completely effaced by extensive lymphocyte infiltrations in the organs from a deceased p80HT transgenic mouse.

B: Immunohistochemical examination of p80HT transgenic mouse lung sections with malignant and lymphocyte infiltration. The sections stained strongly either for B220 (a B-cell marker) or for CD3 (a T-cell marker), indicative of B or T cell lymphomas. Scale bars in (A) and (B), 100 μm.

C: Southern blot analysis of IgH (top) and TCR (bottom) gene rearrangements in representative lymphoma samples. EcoRI-digested DNA Was hybridized with an IgH-μJ_{H4} probe or with a TCR C_{β1} probe. Tail DNA from a p80HT transgenic mouse was used as the control for the germline (GL) IgH and TCR loci. Arrowheads indicate rearrangements at either the IgH-μ or the TCR-β locus. Size markers in kilobases (Kb) are shown to the right.

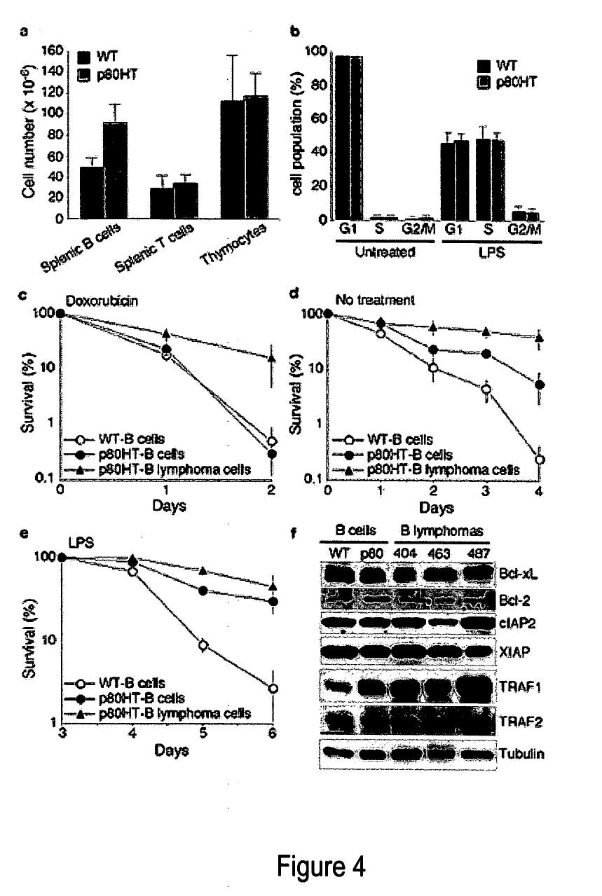

FIG. 4. Lymphocytes and Lymphoma Cells from p80HT Transgenic Mice are Resistant to Certain Apoptotic Stimuli.

A: The numbers of total lymphocytes in the indicated lymphoid organs. Lymphocytes were stained with fluorescence-conjugated antibodies against B220, Thy-1.2, CD4, and CD8, and analyzed by flow cytometry.

B: Cell-cycle analysis of splenic B cells that were either untreated or treated for 48 h with LPS (20 μg/ml). Percentages of cells in each phase of the cell cycle are shown.

C-E: In vitro survival and apoptosis assays of splenic B cells and B lymphoma cells. Cells were either untreated (D) or treated with 0.5 μg/ml of doxorubicin (C) or with 20 μg/ml of LPS (E). Viability was determined by trypan blue dye exclusion assays. Data in (A-E) represent means±SD of cells from 5 mice of each genotype or from 58 lymphomas samples.

F: Immunoblot analysis of the expression of the indicated anti-apoptotic genes in splenic B cells from wild-type and p80HT transgenic mice, and in representative B lymphoma samples. Levels of o-tubulin are shown as the loading control.

### EXAMPLE I

Transgenic mice with Targeted Expression of the NF-_{K}B2 Mutant p80HT Display High Levels of Constitutive _{K}B-Binding Activity in Lymphocytes.

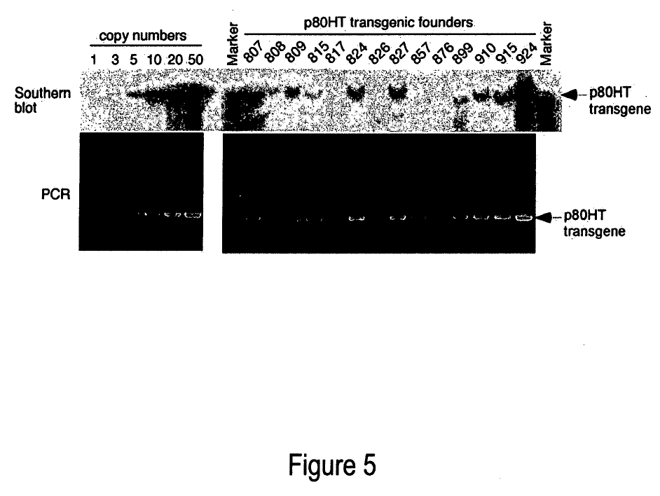

To determine whether NF-_{K}B2 mutations play a role in lymphomagenesis, we generated transgenic mice with targeted expression in both B and T lymphocytes of p80HT, an NF-_{K}B2 mutant originally identified in the human cutaneous T-lymphoma cell line HUT78(Thakur et al, 1994; Zhang et al. 1994). Among the 131 founder mice, 14 (7 males and 7 females) were found to carry various copy numbers of the P80HT trasgene by Southern blot and PCR analyses of tail DNA (FIG. 5). To examine the tissue specific expression of p80HT, two transgenic founder mice and a wild-type littermate were sacrificed and various organs obtained. Immunoblot analysis using a monoclonal antibody against the N-terminal region of human NF-_{K}B2 showed high-level expression of p80HT and p52 (most likely as a result of p80HT processing to p52) only in lymphoid organs of the transgenic mice (FIG. 1B). We also confirmed p80HT expression in purified splenic B and T cells as well as in bone marrow cells (FIG. 1B). Moreover, electrophoretic mobility-shift assays (EMSA) showed that nuclear extracts from unstimulated splenocytes of p80HT transgenic mice had significantly higher levels of constitutive _{K}B-binding activity when compared with the extracts from the wild-type littermate, and the majority of the _{K}B-binding complexes in the p80HT nuclear extracts could be supershifted by an antibody against human NF-_{K}B2 (FIG. 1C). Thus, targeted expression of p80HT in mice resulted in a marked increase in the nuclear KB-binding activity in lymphocytes.

### EXAMPLE II

P80HT Transgenic Mice Develop Lymphomas with Multi-Organ Metastases

To assess the role of p80HT in tumorigenesis, we monitored the remaining 12 transgenic founder mice for tumor development (Table I). Half of them died between 41 to 89 weeks, whereas only one of the 6 wild-type littermates died at the age of 87 weeks. Autopsy and histopathological examinations revealed that all the 6 deceased p80HT founder mice developed lymphomas with extensive metastases in the liver, lung and/or kidney. The deceased wild-type mouse had localized lymphoma. The rest of p80HT founder mice (n=6) and their wild-type littermates (n−5) were sacrificed by 96 weeks of age, and histopathological examinations showed the development of disseminated lymphomas in 5 p80HT mice and of localized lymphoma in one wild-type mouse. The significantly higher tumor incidence in the p80HT transgenic founder mice provides direct evidence that the lymphoma-associated NF-_{K}B2 mutant has an oncongenic activity in vivo. The observation that 11 of the 12 independent p80HT transgenic founders developed lymphomas also rules out the possibility that the tumorigenesis might result from insertional effects of the transgene.

TABLE 1
Lymphoma development in p80HT transgenic founder mice

| | Lifespan | Lymphoid organs | Other organs |
|---|---|---|---|
| Founders | (weeks) | involved | involved |
| P80HT | | | |
| 808 | 41 | Lymph nodes, spleen | Lung, liver |
| 899 | 57 | Lymph nodes, spleen | Lung, liver, |
| | | | kidney |
| 817 | 63 | Lymph nodes, spleen, | Lung, liver, |
| | | thymus | kidney |
| 826 | 87 | Lymph nodes, spleen | Kidney |
| 924 | 88 | Lymph nodes, spleen | Lung, liver |
| 809 | 89 | Lymph nodes, spleen, | Lung, kidney |
| | | thymus | |
| 807 | 96 (killed) | Lymph nodes, spleen | Liver, kidney |
| 815 | 96 (killed) | Spleen | Lung |
| 857 | 96 (killed) | Spleen | Liver |
| 876 | 96 (killed) | No | No |
| 910 | 96 (killed) | Lymph Nodes | Lung |
| 915 | 96 (killed) | Spleen | Liver |
| Wild Type | | | |
| 834 | 87 | Lymph nodes, spleen | No |
| 816 | 96 (killed) | No | No |
| 828 | 96 (killed) | Lymph nodes | No |
| 829 | 96 (killed) | No | No |
| 830 | 96 (killed) | No | No |
| 858 | 96 (killed) | No | No |

To confirm the tumorigenic activity of p80HT in a large-scale study and also to determine the effect of its expression levels on the tumor incidence, we monitored the F2 offspring of two independent p80HT transgenic lines for lymphoma development. Mice from the 808 line expressed significantly higher levels of p80HT in lymphocytes than those from the 815 line (FIG. 2A). Correlated with the p80HT expression levels, the 808 line had a much higher mortality rate in comparison with the 815 line (79% for the 808 line versus 24% for the 815 line during the first 70 weeks, FIG. 2B). All of the wild-type littermates were alive and apparently healthy by 70 weeks. Autopsy examinations revealed that most of the deceased p80HT mice (75%) had lymphadenopathy and splenomegaly (FIGS. 2C and 2D) with extensive metastases in lung, liver, and/or kidney (FIG. 3A).

Histopathological studies were performed on various tissue samples from the deceased p80HT mice. The examination demonstrated that all the animals developed lymphomas with similar pathological features, including complete effacement of normal organ architecture by massive infiltration of small- to medium-sized lymphocytes with pheomorphic nuclei and abundant cytoplasm (FIG. 3A). Immunohistochemical staining of the lung sections with malignant lymphocyte infiltration revealed that these lymphocytes expressed either B220 (a B-cell marker) or CD3 (a T-cell marker) (FIG. 3B), indicating that p80HT transgenic mice developed either B or T cell lymphomas. Of the 10 samples that have been examined so far, 8 were stained strongly for B220 and two for CD3. Thus, the majority of p80HT transgenic mice developed B cell lymphomas.

We next conducted Southern blot analysis of antigen-receptor gene rearrangements to determine the clonality of lymphomas developed in p80HT transgenic mice. Rearrangements at the IgH μ locus were detected in 6 of the 8 lymphoma samples that have been analyzed so far, with the remaining two showing rearrangements at the T-cell receptor β gene locus (FIG. 3C). These results demonstrate that the lymphomas in p80HT mice resulted from clonal expansion of malignant B or T cells.

### EXAMPLE III

Perturbation of B Cell Homeostasis in p80HT Transgenic Mice

To gain insights into the mechanism by which p80HT induces lymphomagenesis, we first assessed the effect of p80HT expression on lymphocyte development. These studies were conducted with 4- to 6-week old transgenic mice from the 808 line and their age-matched wild-type littermates. Immunoflourescent staining and flow cytometry analysis of lymphocytes revealed no significant difference in the numbers of total thymocytes and splenic T cells between p80HT transgenic mice and their wild-type littermates (FIG. 4A). Also, p80HT mice showed normal ratios of the major subsets of thymocytes (CD4⁻8⁻, CD4⁺8⁺, and CD4⁻8+ and of splenic T cells (CD4⁺ and CD8⁺)(data not shown). However, p80HT transgenic mice displayed a marked increase (87%) in the number of total splenic B cells in comparison with the wild-type littermates (FIG. 4A), demonstrating that p80HT expression resulted in expansion of the B cell population. This finding is consistent with the observation that most of the lymphomas developed in p80HT mice were of B cell origin.

### EXAMPLE IV

B Lymphocytes from p80HT Transgenic Mice Show no Defects in Proliferation Responses but are Resistant to Certain Apoptotic Stimuli

The accumulation of splenic B cells in p80HT transgenic mice could result from excess production of mature B cells in the bone marrow, increased proliferation or reduced cell death of mature B cells, or a combination of these factors. Flow cytometry analysis revealed no abnormality in the number of mature B cells (B220⁺ sigM⁺) in the bone marrow of p80HT mice. We also performed colony-forming unit (CFU) assays of bone marrow pre-B cells, and ob served no difference in the CFU numbers between the transgenic mice and wild-type littermates (data not shown). These results suggest that p80HT expression has no significant effect on the production and development of B cells in the bone marrow.

We next examined the growth properties of splenic B cells from 4- to 6-week-old p80HT transgenic mice. Freshly isolated B cells showed no significant proliferation, as determined by cell cycle analysis (FIG. 4B). To assess whether p80HT expression enhances the proliferative responses of B cells to mitogenic stimuli, purified splenic B cells were treated either with the polyclonal mitogen LPS (lipopolysaccharide) or with an anti-μchain antibody F(ab′)₂, which induces ligation of the surface IgM (sigM). After 2-day treatment, the cells were collected and subjected to cell cycle analysis. The percentages of cells in the S and G₂/M phases were similar between p80HT and wild-type B cells (FIG. 4B). We also performed ³H-thymidine incorporation assays and observed no significant difference in the levels of ³H-thymidine incorporation between mitogen-stimulated p80HT and wild-type B cells. Together, these data indicate that p80HT expression does not render B cells to grow autonomously or enhance their proliferative response to mitogenic stimuli. Thus, the observed accumulation of B cells in p80HT transgenic mice is not due to increased proliferation.

Apoptosis also plays a critical role in maintaining lymphocyte homeostasis (Rathmell and Thompson, 2002). As p80HT is an anti-apoptotic protein (Wang et al., 2002), its overexpression may enhance B cells survival, leading to other observed increase in the B cell population. His possibility was investigated by comparing the sensitivity of splenic B cells from p80HT transgenic mice and wild-type littermates to a variety of death stimuli including cytokine deprivation, death ligands, DNA damage, and mitogenic stimulation. Both wild-type and p80HT B cells were highly resistant to Fas ligand an TNF-a. These cells were also equally sensitive to apoptosis induced by the DNA damage drug doxorubicin (FIG. 4C). However, p80HT splenic B cells showed markedly enhanced survival in the absence of cytokines (FIG. 4D). Moreover, during the course of investigation we noted that in response to LPS, splenic B cells generally proliferated for about 3 days and then underwent extensive apoptosis. Although P80HT B cells displayed a normal proliferative response to LPS (FIG. 4B), they were highly resistant to apoptosis induced by the activation signal (FIG. 4E). Thus, p80HT specifically promotes survival of B cells in the absence of growth cytokines or following mitogenic activation, which most likely contributes to the B cell expansion observed in the transgenic mice.

To further assess the role of apoptosis suppression in lymphoma development in p80HT transgenic mice, we examined apoptotic response of B lymphomas cells in comparison with their pre-tumor counterparts. In contrast to p80HT pre-tumor B cells, the lymphoma cells were highly resistant to apoptosis induced by the DNA-damage drug doxorubicin (FIG. 4C). The tumor cells also survived better in the absence of cytokines (FIG. 4D). These results show that additional genetic or epigenetic alterations took place during the course of tumor igenic transformation to facilitate the survival and expansion of malignant B cell clones.

### EXAMPLE V

Upregulation of TRAF Proteins in Lymphocytes and Lymphoma Cells From p80HT Transgenic Mice

P80HT can bind KB sites in its unprocessed form and transactivate reporter genes through heterodimerization with RelA (Chang et al., 1995). Human lymphoma cell lines carrying p80HT by chromatin immunoprecipitation, RNase protection assay, and immunoblotting indicated that p80HT targets several anti-apoptotic genes from upregulation, including TRAF1 (TNF receptor-associated factor 1), TRAF2, clAP2 (cellular inhibitor of apoptosis 2), and Bcl-X_{L}. Therefore, we examined the levels of these and other related anti-apoptotic proteins in splenic B cells from wild-type and p80HT mice, as well as in p80HT lymphoma cells. Immunoblot analysis revealed no difference in the levels of Bcl-X_{L}, Bcl-2, clAP2, and XIAP between these cells (FIG. 4F). However, pre-tumor B cells and B lymphoma cells from p80HT transgenic mice showed a significant increase in the levels of TRAF1 and, to a less extent, of TRAF2 as well, in comparison with B cells from wild-type littermates (FIG. 4F). These results suggest that the TRAF genes are the major targets of p80HT in its induction of lympomagenesis in mice.

### Results

This invention directly demonstrates a relationship between NF-_{K}B2 mutations and lymphomagenesis in mice. We further show that p80HT expression has no effect on the proliferation of lymphocytes but renders them resistant to certain apoptotic stimuli, leading to a marked increase in the B cell population. Thus, the anti-apoptotic activity of p80HT is critical for its oncogenic function. These findings suggest a model for the B cell lymphoma development in p80HT transgenic mice. Expression of p80HT, which results in an increase in the nuclear _{K}B-binding activity, confers a survival advantage to B cells in the absence of growth cytokines and following mitogenic stimulation, probably through the TRAF1 anti-apoptotic pathway. This survival advantage facilitates clonal growth of the B cells stimulated by repetitive exposure to microbes and to autoantigens. Some of these B cells clones accumulate additional genetic or epigenetic lesions during the expansion process and become transformed.

There is convincing evidence for an essential role of NF-_{K}B activity in cellular transformation by some oncogenes (Finco et al., 1997; Reuther et al., 1998) and in inflammation-associated cancer development (Greten et al., 2004; Pikarsky et al., 2004). However, it is much less clear whether a sustained activation of NF-_{K}B activity per se is an oncogenic event in vivo. Transgenic mice with targeted expression of Rel proteins in thymocytes do not develop thymic or peripheral T-cell lymphomas (Perez et al., 1995; Weih et al., 1996), I_{K}Bα-deficient mice die 7-10 days post-natally (Beg et al., 1995; Klement et al., 1996), which prevents an examination of the potential tumorigenic effect of I_{K}Bα inactivation. Mice deficient in I_{K}Bα, which is expressed at high levels in thymocytes and periperhal T cells, show no obvious phenotype (Memet et al., 1999). Thus, this invention along with the recent report of mammary tumor development in transgenic mice with targeted expression of c-Rel (Romieu-Mourez et al., 2003), firmly establishes that constitutive activation of NF-_{K}B can directly initiates tumorigenesis.

In normal cells, the NF-_{K}B1 p50-RelA activity is under the control of I_{K}B-factors, whereas the NF-_{K}B2 p52-RelB activity is liberated by degradation of the I_{K}B-like sequence in the C-terminal region of NF-_{K}B2 p100 (Karin et al., 2002). We suggest that these different modes of regulation of NF-_{K}B1- and NF-_{K}B2 associated activities underlie the apparently distinct mechanisms for the sustained activation of NF-_{K}B in oncongenic process. Deregulation of IKK activity appears to be the preferred mode for the activation of NF-_{K}B1 p50-RelA in human cancers, as exemplified by MALT lymphomas (Lucas et al., 2001; Ruland et al., 2001; Uren et al., 2000) and inflammation-associated cancers (Greten et al., 2004; Pikarsky et al., 2004). This model is also consistent with genetic evidence. Alterations at the human NF-_{K}B1 and RelA loci on chromosomes 4q24 and 11q13, respectively, are rarely found in leukemias and lymphomas (Karin et al., 2002; Rayet and Gelinas, 1999). Moreover, transgenic mice with targeted expression of RelA in thymocytes display a corresponding increase in endogenous I_{K}Bα (Perez et al., 1995), which is expected to retain the NF-_{K}B1 p50-RelA complex in the cytoplasm. Thus, constitutive activation of IKK would be more effective in disruption of the I_{K}B-mediated negative feedback control of NF-_{K}B1 p50-RelA activity. The C-terminal I_{K}B-like domain of NF-_{K}B2 p100 functions as a major I_{K}B activity in the control of NF-_{K}B2-RelB activity (Solan et al., 2002). Therefore, C-terminal deletions and rearrangements at the NF-_{K}B2 locus, which result in removal of various portions of the C-terminal I_{K}B-like sequence of p100 (FIG. 1A), would be an effective way for the activation of NF-_{K}B2 activity, leading to lymphoma development.

The NF-_{K}B2 signaling pathway plays a critical role in the maintenance of the peripheral B cell population (Caamano et al., 1998; Franzoso et al., 1998). The cytokine BAFF (B-cell activating factor, also known as BLyS), which is required for the development and survival of peripheral B cells Schiemann et al., 2001), activates NF-_{K}B2 by inducing processing of P100 to generate p52 (Claudio et al, 2002; Kayagaki et al., 2002). P80HT transgenic mice express high levels of p52 in lymphocytes (FIG. 1B), probably as a result of constitutive processing of p80HT to p52 (Xiao et al., 2001). Thus, it is formally possible that the observed B cell expansion and lymphomagenesis in p80HT mice is a consequence of the sustained activation of p52. This possibility is currently under investigation in p52 transgenic mice.

TRAF1 and TRAF2 are members of the TRAF family of adapter proteins that interact with and integrate signals from several members of the TNF receptors (TNFR) family, and play an important role in regulation of diverse cellular processes, including apoptosis (Arch et al., 1998). Notably, a number of recent studies implicate TRAF1 in the pathogenesis of lymphoid malignancies. TRAF1 is overexpressed in a variety of lymphoma and leukemia cell lines and specimens, such as Hodgkin's and non-Hodgkin's lymphomas, and B-cell chronic lymphocytic leukemia (Durkop et al., 1999; Izban et al., 2000; Munzert et al., 2002; Murray et al., 2001; Savage et al., 2003; Zapata et al., 2000). Also, association with TRAF proteins, including TRAF1, is critical for the Epstein-Barr virus-encoded latent membrane protein 1 to transform primary B cells (Cahir McFarland et al., 1999). Moreover, transgenic mice expressing a TRAF2 mutant lacking the N-terminal RING and zinc finger domains (TRAF2DN), which structurally mimics TRAF1, display a polyclonal expansion of B cells (Lee et al., 1997), and TRAF2DN cooperates with Bcl-2 to induce B lymphoma and leukemia in transgenic mice (Zapata et al., 2004). Our study further links NF-_{K}B2 mutations to TRAF1 activation. Together, these findings suggest that that TRAF1 is a crucial mediator of diverse oncongenic signals in the development of many lymphoid malignancies.

The p80HT transgenic mice are the first animal model for human leukemias and lymphomas that carry mutations in the NF-_{K}B2 gene. These mice should be useful for the identification of genes and signaling pathways that cooperate with NF-_{K}B2 mutations in the pathogenesis of lymphoid malignancies, and for the development and testing of therapeutic and prevention drugs that specifically interfere with the pathogenic process.

### EXAMPLE VI

Generation of p80HT Transgenic Mice

The coding sequence for human p80HT was amplified by PCR using the human fetus Marathon-ready cDNA library (Clontech) as the template, with specific oligonucleotide primers designed on the basis of the sequence of GenBank accession number U09609 (Thakur et al., 1994). The DNA fragment was inserted into the Sa/I and BamHI sites of the pHSE3 expression vector. The vector contains an H-2 Kb promoter at the 5′ end and an immunoglobulin heavy chain (μ) enhancer at the 3′ end that targets transgene expression specifically in lymphocytes (Pircher et al., 1989; Zhang et al., 2002). The plasmid was linearized by Pvul and microinjected into the male pronuclei of fertilized eggs derived from C57BL/6J×SJL/J F2 mice (University of Michigan Transgenic Animal Model Core). Transgenic founders were identified by the Southern blot analysis of BamHI digested tail DNA using p80HT cDNA as the probe and by PCR amplification of a 1.3 kb product using a set of primers derived from p80HT cDNA: 5NFKBsal (5′-GCG TTC GAC ATG GAG AGT TGC TAC MC CCA G-3′) and 3NFKBp52 (5′-GCG GGA TCC TCA TCG CTG CAG CAT CTC CGG GGC-3′). 14 Transgenic founders were obtained. Two independent liens 808 and 815 were established by mating male founders (#808 and #815) to C57BL/6J×SJL/J F1 female. Transgenic founders and transgenic progenies of the 808 and 815 lines were monitored daily for tumor development with their wild-type littermates as control. All mice were maintained in specific pathogen free room at the Animal Facility of the Medical College of Ohio in accordance with the institutional guidelines. Electrophoretic mobility shift assays (EMSA) Nuclear extracts were prepared from splenocytes using the NE-PER Nuclear Extraction kit according to the manufacturer's protocol (Pierce). EMSA were performed as previously described (Wang et al. 2002), except that 3 μg of nuclear extracts were incubated with 2 μl of either preimmune rabbit serum or an antiserum against p52 (Upstate) in the binding buffer for 1.5 h at 4° C. before addition of the KB probe.

Immunoblot

Cells were directly suspended in the standard SDS sample buffer. Protein concentrations were determined with the Bio-Rad protein assay kit, using bovine serum albumi as reference. 50 μg of proteins were separated on 10% or 12% SDS-polyacrylamide gels and transferred to nitrocellulose membranes, which were then probed with antibodies and visualized by ECL. The following primary antibodies and dilutions were used: muse anti-human NF-_{K}B2 p52, 1:500 (Upstate); rabbit anti-Bcl-2, 1:100 (Santa Cruz); rabbit anti-BCl-x_{L}, 1:200 (Santa Cruz); rabbit anti-clAP2, 1:200 Santa Cruz); rabbit anti-XIAP, 1:500 (Cell Signaling); rabbit anti-TRAF1, 1:200 (Santa Cruz); rabbit anti-TRAF2, 1:200 (Santa Cruz); mouse anti-α-tubulin, 1:2000 (Sigma). Horseradish peroxidase-conjugated anti-mouse and anti-rabbit were used as secondary antibodies (ICN).

Flow Cytometry

Single-cell suspensions were prepared from the thymus, spleen, bone marrow, and lymph nodes of 4- to 6-week-old mice according to standard procedures. Red blood cells were lyzed by treatment for 5 min with ACK lysis buffer (150 mM NH₄Cl, 10 mM KHCO₃, 0.1 mM EDTA, pH 7.3), and dead cells were removed by passing through the gradient medium Lympholyte-M (Cedarlane). Lymphocytes were then stained with the following antibodies: fluorescein isothiocyanate (FITC)-conjugated rat anti-B220 and -CD4; R-phycoerythrin (PE)-conjugated rat anti-thy-1.2, -CD8a, and -IgM (BD Pharmingen) and analyzed by flow cytometry (Epics Elite, Beckman-Coulter).

Histopathology and Immunohistochemistry

Tumors and normal tissue samples were fixed in 10% neutral buffered formalin, embedded in paraffin blocks, sectioned at 5 μm, and stained with Hematoxylin and eosin. For immunohistochemistry, the paraffin was removed and sections were rehydrated according to standard procedures. For retrieval of B220 and CD3 antigens, the sections were subject to boiling in 10 mM citrate buffer (pH 6.0) and 1 mM EDTA (pH 8.0) for 10 min, respectively. Following quenching of endogenous peroxidase activity with H₂O₂ and blocking with normal serum, the sections were incubated for 60 min with either a rat anti-mouse CD45R/B220 monoclonal antibody (5 μg/ml, BD Pharmingen), a rat anti-human CD3 monoclonal antibody (10 μg/ml, BD Pharmingen). After washing, a biotinylated rabbit anti-rat secondary antibody (Vector Laboratories) was applied for 30 min. The sections were then incubated for 30 min with ABC reagent (Vector Laboratories), and the immunostaining was visualized with 3,3′-diaminobenzidine (DAB, Sigma). Finally, the tissue sections were counter-stained with hematoxylin and examined under a light microscope.

Southern Blot Analysis of Antigen-Receptor Gene Rearrangements

Genomic DNA was prepared from tails and primary tumor samples, and Southern blot was performed according to standard procedures. Briefly, 10 μg genomic DAN was digested with EcoRI, resolved by 0.8% agarose gel electrophoresis, transferred to nylon membranes, and hybridized with a J_{H4} probe to deal the IgH-p gene rearrangements (Adams et al., 1985) or with a C_{β1} probe to screen for the TCRβ gene rearrangements (Hedrick et al., 1984).

In Vitro Lymphocyte Proliferation Assays

Splenic B cells were purified from 4- to 6-week-old mice with mouse B immunocolumn according to the manufacturer's instructions (Cedarlane). The purified cells were cultured in DMEM supplemented with 10% FBS, 250 μM L-asparagine, and 50 μM 2-mercaptoethanol. B cells (10⁵/well, 96-well plate) were stimulated with 20 μg/ml of LPS (Sigma). After 48 h, cells were harvested for cell cycle analysis on an Epics Elite flow cytometer (Beckman-Coulter). The data were analyzed with MultiCycle AV (Phoenix Flow Systems).

In Vitro Lymphocyte Survival and Apoptosis Assays

Purified splenic B cells and B lymphoma cells (10⁵/well, 96-well plate) were cultured in DMEM supplemented with 10% FBS, 250 μM L-asparagine, and 50 μM 2-mercaptoethanol. Cells were either untreated or treated with recombinant mouse TNF-α (20 ng/ml, Calbiochem), Fas ligand (100 ng/ml, PeoproTech), LPS (20 μg/ml, Sigma), or doxorubicin (0.5 μg/ml, Ben Venue Laboratories), and viable cells were determined daily by trypan blue dye exclusion assays.

FIG. 5 shows analyses of p80HT transgenic mice. (a) Southern blot genotyping. Though not detectable here, PCR analysis indicated that the founder lines 817, 826 and 876 contain one copy of p80HT transgene. Copy of numbers were determined using human p80HT cDNA. (b) Immunoblot analysis showing specific expression in transgenic mouse lymphoid tissues of human p80HT and its processing product, p52. Also shown are levels of a-tubulin as the loading control. The data shows that the mice carry the p80HT transgene detected by two different methods, Southern blotting and PCR. This demonstrates that we have generated the p80HT transgenic mice.

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

We claim:

1. A transgenic mouse having the human NF-_{K}B2 mutant gene p80HT derived from a lymphoma patient.

2. The p80HT transgenic mouse being an animal model for human lymphomas with mutations in the NF-_{K}B2 gene.

3. The transgenic mouse of claim 1 wherein the mouse has B- and T-cell lymphomas.

4. The transgenic mouse of claim 3 wherein the cells are lymphocytes in the lymph nodes and spleen.

5. The transgenic mouse of claim 1 wherein the mouse has extensive metastases in multiple organs.

6. The transgenic mouse of claim 5 wherein the organs are the liver, lung and kidney.

7. The transgenic mouse of claim 5 wherein the cells of metastases are lymphoma cells.

8. The transgenic mouse of claim 2 wherein the mouse can be used for preclinical animal trials of therapeutic drugs for human lymphomas with mutations in the NF-_{K}B2 gene.

9. The transgenic mouse of claim 2 wherein the mouse can be used for preclinical animal trials of preventive drugs for human lymphomas with mutations in the NF-_{K}B2 gene.

10. The transgenic mouse of claim 2 wherein the mouse can be used to reveal the pathophysiology of human lymphomas with mutations in the NF-_{K}B2 gene.

11. The transgenic mouse of claim 3 wherein the lymphoma cells can be used for screening of chemical and biological molecules for the development of therapeutic drugs for lymphomas with mutations in the NF-_{K}B2 gene.

12. The transgenic mouse of claim 3 wherein the lymphoma cells can be used for screening of chemical and biological molecules for the development of preventive drugs for lymphomas with mutations in the NF-KB2 gene.

13. The transgenic mouse of claim 3 wherein the lymphoma cells can be used to reveal the genes, their protein products and molecular processes essential for the development of lymphomas with mutations in the NF-_{K}B2 gene.

14. A method of generating a p80HT transgenic mouse comprising the steps of:
a) introducing the NF-KB2 p80HT gene into a mouse embryonic cell;
b) generating a transgenic mouse from the cells of step a);
c) breeding the transgenic mouse having the p80HT gene; and
d) demonstrating that the transgenic mouse is a p80HT mouse by genetic, biochemical, and functional analyses.

15. A method of determining an expression pattern of a cell sample independent of the proportion of sub mucosal, smooth muscle, or connective tissue cells present, comprising the steps of:
a) taking tissue samples from a p80HT transgenic mouse;
b) determining expression of the p80HT gene in the samples comprising tissue cells, wherein the gene excludes other genes expressed in the tissue; and
c) displaying a pattern of expression of the p80HT gene for the samples which are independent of the proportion of tissue cells in the samples.
